(19) United States
(12) Patent Application Publication
(10) Pub. No.: US 2025/0040884 A1
(43) Pub. Date: Feb. 6, 2025

(54) DEVICE AND METHODS FOR MONITORING AND TRAINING BREATHING

(71) Applicants: Breezee, Inc., Crestwood, KY (US); Liberate Medical LLC, Crestwood, KY (US)

(72) Inventors: Cedric Francois, Crestwood, KY (US); Angus McLachlan, Louisville, KY (US)

(73) Assignees: Breezee, Inc., Crestwood, KY (US); Liberate Medical LLC, Crestwood, KY (US)

(21) Appl. No.: 18/718,237

(22) PCT Filed: Dec. 16, 2022
(86) PCT No.: PCT/US2022/053210

§ 371 Date: Jun. 10, 2024

Related U.S. Application Data
(60) Provisional application No. 63/290,465, filed on Dec. 16, 2021.

(51) Int. Cl. A61B 5/00; A61B 5/08; A61B 5/097

(52) U.S. Cl. CPC A61B 5/486; A61B 5/0803; A61B 5/097; A61B 5/6803; A61B 5/7271; A61B 5/742; A61B 2560/045; A61B 2560/0462; A61B 2562/0247

(57) ABSTRACT

A breath monitoring apparatus is provided herein, the apparatus having one or more respiration sensors configured to output a respiration signal indicative of a respiration parameter of a user during a respiration activity: a feedback indicator configured to provide a sensory output to a user relating to the respiration signal; and a controller communicatively coupled to the respiration sensor and the feedback indicator, and configured to: provide instructions correlating to a desired breathing activity to the user during the respiration activity: receive and analyze the respiration signal output by the respiration sensor to determine a biofeedback score based on the respiration signal, wherein the biofeedback score is indicative of the user's respiration signal compared to the desired breathing pattern; and provide feedback to the user through the feedback indicator based on the biofeedback score, wherein the breath monitoring apparatus is configured to be wearable by the user.

### CROSS REFERENCE TO RELATED APPLICATION

This application claims benefit of priority of U.S. Provisional Patent Application No. 63/290,465, filed Dec. 16, 2021, entitled, “DEVICE AND METHODS FOR MONITORING AND TRAINING BREATHING.” the content of which is hereby incorporated by reference in its entirety.

### TECHNICAL FIELD

The present disclosure relates to the field of breathing monitors. Specifically, the disclosure relates to devices, systems, and methods for monitoring and observing breathing in real time and training breathing in a subject.

### BACKGROUND

Evidence has suggested that feedback training has proven beneficial in the treatment of COPD. Training patients to take longer exhalations can improve exercise tolerance and improve symptoms like dyspnea. Additionally, some activities, such as yoga, Pilates, and tai-chi have specialized breathing techniques. In these activities, it is desired that a person exhale, inhale, and/or hold their breath at correct points during the practice. It could also be useful to guide breathing meditation or general wellness. For example, studies have shown that slow breathing training may reduce blood pressure, improve heart rate variability and other parameters. Accordingly, a need exists for a system and device to monitor breathing and provide feedback to the user in real time.

### SUMMARY

Provided herein are systems, devices, and methods for that monitor breathing patterns of a user in real-time and provide feedback to the user. In operation, the system may collect information about the user's breathing. This information can be recorded, analyzed and tracked over time to identify patterns or changes. Other operations may guide a user to a desired breathing pattern as well as measure and analyze the user's actual breathing pattern compared to the desired breathing pattern.

In one embodiment, a breath monitoring apparatus is provided, the breath monitoring apparatus having one or more respiration sensors configured to output a respiration signal indicative of a respiration parameter of a user during a respiration activity; a feedback indicator configured to provide a sensory output to a user relating to the respiration signal; and a controller communicatively coupled to the respiration sensor and the feedback indicator. The controller is configured to provide instructions to the user during the respiration activity, wherein the instructions correlate to a desired breathing pattern; receive the respiration signal output by the respiration sensor; analyze the respiration signal to determine a biofeedback score based on the respiration signal detected after the instructions, wherein the biofeedback score is indicative of the user's respiration signal compared to the desired breathing pattern; and provide feedback to the user through the feedback indicator based on the biofeedback score. The breath monitoring apparatus is configured to be wearable by the user.

In another embodiments, a system for monitoring respiration is provided, having a respiration sensor configured to output a respiration signal indicative of a respiration parameter of a user during a respiration activity; a feedback indicator configured to provide a sensory output to a user relating to the respiration signal; and a controller coupled to the respiration sensor and the feedback indicator, the controller configured to: provide instructions to the user during the respiration activity, wherein the instructions correlate to a desired breathing pattern; receive the respiration signal detected by the respiration sensor; analyze the respiration signal to determine a biofeedback score based on the respiration signal detected after the instructions, wherein the biofeedback score is indicative of the user's respiration signal compared to the desired breathing pattern; and provide feedback to the user through the feedback indicator based on the biofeedback score.

In another embodiment, a method for monitoring respiration using the apparatus and/or system of any of the aspects disclosed herein is provided, the method comprising providing instructions to a user during a respiration activity, wherein the instructions correlate to a desired breathing pattern; sensing a respiration parameter using a respiration sensor configured to output a respiration signal indicative of the respiration parameter of the user during the respiration activity; analyzing the respiration signal to determine a biofeedback score based on the respiration signal detected after the instructions, wherein the biofeedback score is indicative of the user's respiration signal compared to the desired breathing pattern; and providing feedback to the user through a feedback indicator configured to provide a sensory output to the user based on the biofeedback score.

These and other objects, features, embodiments, and advantages will become apparent to those of ordinary skill in the art from a reading of the following detailed description and the appended claims.

### BRIEF DESCRIPTION OF THE DRAWINGS

FIG. 1 depicts a schematic view of a system for monitoring breathing in real-time, according to one or more embodiments shown and described herein;

FIG. 2A depicts the system of FIG. 1 in a wearable breath monitoring apparatus, according to one or more embodiments shown and described herein;

FIG. 2B depicts a perspective view of the tubing system of the breath monitoring apparatus of FIG. 2A, according to one or more embodiments shown and described herein;

FIG. 2C depicts a cross-sectional view of the tubing system of FIG. 2B, according to one or more embodiments shown and described herein;

FIG. 2D depicts a cross-sectional view of the electronics housing of the breath monitoring apparatus of FIG. 2A, incorporating the system of FIG. 1, according to one or more embodiments shown and described herein;

FIG. 3 depicts a flowchart illustrating a method for monitoring a respiration activity, according to one or more embodiments shown and described herein;

FIG. 4A depicts a graph illustrating a method of analyzing the data collected, according to one or more embodiments shown and described herein;

FIG. 4B depicts a graph illustrating data collected using nasal and oral breathing parameters, according to one or more embodiments shown and described herein;

FIG. 4C depicts graph illustrating data collected interpreting inhalation and exhalation, according to one or more embodiments shown and described herein; and

FIG. 4D depicts an analysis of the data presented in FIG. 4C comparing the actual breathing parameters with the target breathing pattern, according to one or more embodiments shown and described herein.

### DETAILED DESCRIPTION

The details of embodiments of the presently-disclosed subject matter are set forth in this document. Modifications to embodiments described in this document, and other embodiments, will be evident to those of ordinary skill in the art after a study of the information provided in this document.

While the following terms are believed to be well understood in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in this specification and the appended claims, the singular forms “a.” “an.” and “the” include plural references unless the content clearly dictates otherwise.

Embodiments of the present disclosure are generally directed to systems that include wearable breathing devices that monitor breathing patterns in real-time and may provide feedback to the user. In operation, the system may collect information about the user's breathing. This information can be recorded, analyzed and tracked over time to identify patterns or changes. Other operations may guide a user to a desired breathing pattern as well as measure and analyze the user's actual breathing pattern compared to the desired breathing pattern.

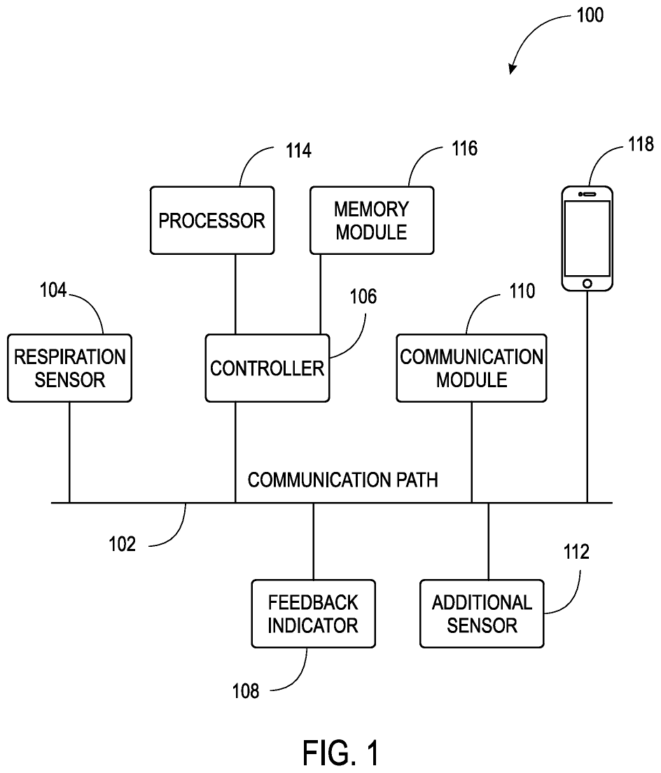

Referring now to the drawings, FIG. 1 depicts a system 100 for monitoring breathing. In embodiments, the system 100 includes a communication path 102, one or more respiration sensors 104, a controller 106, and a feedback indicator 108. As shown in FIG. 1, the controller 106 also include one or more processors 114 and a memory module 116. In embodiments, the system 100 also includes one or more additional sensors 112. The system 100 may further include a communication module 110. In embodiments, the system 100 also includes an external device 118.

As described above, the system 100 includes a communication path 102 that provides data interconnectivity between various modules disposed within the system 100. Specifically, each of the modules operates as a node that is configured to send and/or receive data. The communication path 102 communicatively couples the various components of the system 100. As used herein, the term “communicatively coupled” means that coupled components are capable of exchanging data signals with one another such as, for example, electrical signals via conductive medium, electromagnetic signals via air, optical signals via optical waveguides, and the like. Additionally, it is noted that the term “signal” means a waveform (e.g., electrical, optical, magnetic, mechanical or electromagnetic), such as DC. AC, sinusoidal-wave, triangular-wave, square-wave, vibration, and the like, capable of traveling through a medium.

In embodiments, the communication path 102 includes a conductive material that permits the transmission of electrical data signals to processors, memories, controllers, and sensors throughout the system 100. The communication path 102 may be formed from any medium that is capable of transmitting a signal such as, for example, conductive wires, conductive traces, optical waveguides, or the like. In embodiments, the communication path 102 is at least one wire that communicatively couples the controller 106 to the respiration sensor 104 and/or the feedback indicator 108. In some embodiments, the communication path 102 facilitates the transmission of wireless signals, such as Wifi, Bluetooth, and the like. Moreover, the communication path 102 may be formed from a combination of mediums capable of transmitting signals. For example, the communication path 102 may comprise a combination of conductive traces, conductive wires, connectors, and buses that cooperate to permit the transmission of electrical data signals to components such as processors, memories, sensors, input devices, output devices, and communication devices. Accordingly, the communication path 102 may comprise a vehicle bus, such as for example a LIN bus, a CAN bus, a VAN bus, and the like.

As described above, the system 100 further includes one or more respiration sensors 104. The respiration sensor 104 may be any sensor capable of collecting physiological data and producing a signal indicative of one or more respiration parameters of the user. Illustrative, non-limiting examples of the respiration sensor 104 include pressure sensors, pressure transducers, microphones, thermistors, thermocouples, respiratory inductive plethysmography, piezoelectric belts, electromyogram sensors, mechanomyogram sensors, and the like. In embodiments, the controller 106 is configured to receive data from the one or more respiration sensors 104 via the communication path 102. Types of information that are capable of being calculated from the breathing signal include, but are not limited to: nasal inhalatory breath flow, nasal exhalatory breath flow, oral inhalatory breath flow, oral exhalatory breath flow, blood gas concentration, tidal CO2, flow profile, breathing phase (e.g., inspiration, inspiratory hold, exhalation, expiratory hold), phase transition, inspiratory time, expiratory time, inspiratory and/or expiratory hold time, total breath period, ratio of inspiratory time to breath period, respiratory rate, respiratory rate variability, peak amplitude during exhalation, peak amplitude during inhalation, mean amplitude during exhalation, mean amplitude during inhalation, area under the curve for inhalation, area under the curve for exhalation, and the like. Other respiratory parameters can be calculated depending on the type and/or number of sensors used. For example, with a respiratory inductive plethysmograph, the contribution of abdominal and chest breathing could be calculated. Two or more respiration sensors 104 could be used to calculate the percentage or contribution of oral breathing compared with nasal breathing. Other calibrated sensors may allow the breath volumes and flow rates to be calculated.

In embodiments, the one or more respiration sensors 104 includes any number of desired sensors as required for measuring the intended signal. For example, in embodiments, the system includes 1, 2, 3, 4, 5, 6, 7, 8, 9, or 10 respiration sensors 104, though additional respiration sensors 104 may be included without departing from the scope of the present disclosure. In some embodiments, the system include two respiration sensors 104. In embodiments, the respiration sensors 104 are pressure transducers, configured to detect a pressure change as a result of a respiration activity.

The system 100 further includes one or more feedback indicators 108. In embodiments, the feedback indicator 108 is communicatively coupled to the respiration sensor 104 and/or the controller 106 by the communication path 102. The feedback indicator 108 is configured to provide feedback to the user. In embodiments, the feedback provided by the feedback indicator 108 is a sensory output relating to the respiration signal output by the respiration sensor 104. In embodiments, feedback is provided as real time feedback and/or after completion of a training program, described in further detail below.

In embodiments, the sensory output includes audio feedback, visual feedback, tactile feedback and/or combinations thereof. In embodiments the feedback provided is auditory feedback. In embodiments, the feedback provided is visual feedback. In embodiments, the feedback provided is tactile feedback. In embodiments the feedback includes both auditory and visual feedback. In embodiments the feedback includes both auditory and tactile feedback. In embodiments, the feedback includes both visual and tactile feedback. In embodiments the feedback includes auditory, visual, and tactile feedback.

In embodiments, the feedback indicator 108 provides audio feedback via headphones, speakers, bone conduction and the like. Audio feedback can include, but is not limited to, chimes, dings, vocal cues, musical changes, etc. In embodiments, the feedback indicator 108 provides visual feedback as the sensory output. Visual feedback can be delivered via digital display (e.g., computer screen, smartwatch display, smart phone, tablet, etc.), light signals controlled by an LED, a light tube, a VR headset, an external device configured to change color and/or lighting pattern, and the like. In embodiments, the feedback indicator 108 provides tactile feedback as the sensory output. Tactile feedback can be delivered via vibration, such as with a smartwatch or smart phone or an electronics housing configured to vibrate (e.g., via a vibrating motor), smart fabrics that compress the body based on an electrical signal (e.g., electroactive polymers), electrical stimulation (delivered either to cause muscle contraction or sensory stimulation), and/or magnetic stimulation. In embodiments, tactile feedback is provided to the breathing muscles. (e.g., to inspiratory muscles during inspiration, expiratory muscles during exhalation) to reinforce feedback loops.

In embodiments, the feedback indicator 108 may be provided in an external device 118, discussed in further detail below.

Still referencing FIG. 1, the system 100 includes a controller 106. In embodiments, the controller 106 is communicatively coupled to the respiration sensor 104 and/or the feedback indicator 108 by the communication path 102. In embodiments, the controller 106 is also communicatively coupled to other components of the system 100. For example, in some embodiments the controller 106 can be wired to the respiration sensor 104 and/or the feedback indicator 108. In other embodiments, the controller 106 is communicatively coupled to the other modules of the system 100 via wireless means. In embodiments, the controller 106 is configured to control various operations of the system 100.

In embodiments, the controller 106 is implemented using integrated and/or discrete hardware elements, software elements, and/or a combination thereof. Examples of integrated hardware elements include, but are not limited to, processors, microprocessors, microcontrollers, integrated circuits, application specific integrated circuits (ASIC), programmable logic devices (PLD), digital signal processors (DSP), field programmable gate arrays (FPGA), logic gates, registers, semiconductor devices, chips, microchips, chip sets, microcontrollers, system-on-chip (SoC), and/or system-in-package (SIP). Examples of discrete hardware elements include, but are not limited to, circuits and/or circuit elements such as logic gates, field effect transistors, bipolar transistors, resistors, capacitors, inductors, and/or relays. In some embodiments, the controller 106 includes a hybrid circuit comprising discrete and integrated circuit elements or components. By executing instruction code stored in the memory module 116, the controller 106 may control various components of the system 100, such as the feedback indicator 108, and/or a user display, for example. In embodiments, the controller 106 is configured to transmit data to an external device 118. In embodiments, the data is transmitted using the communications module 110. In embodiments, the data is the signal output from the one or more respiration sensors 104. In embodiments, the data is analyzed and/or processed by the controller 106 before transmission. In embodiments, the controller 106 is embedded into an external device 118 and the signal from the respiration sensor 104 is transmitted by the communication module 110 before analysis and/or processing.

As noted above, in embodiments, the controller 106 includes one or more processors 114. The one or more processors 114 of the system 100 may include any device capable of executing machine-readable instructions. In embodiments, the one or more processors 114 may be communicatively coupled to the other components of system 100 by the communication path 102.

Computer-executable instruction or firmware implementations of the processor 114 may include computer-executable or machine-executable instructions written in any suitable programming language to perform the various functions described. As used herein, the term “processor” includes any microprocessor, microcontroller, or other basic computing device that incorporates the functions of a computer's central processing unit (CPU) on an integrated circuit. Accordingly, the one or more processors 114 may be an integrated circuit, a microchip, a computer, or any other computing device.

In addition to the processor 114, the controller 106 further includes one or more memory modules 116. In embodiments, the one or more processors 114 are a programmable device that receives the respiration signal from the respiration sensor 104, processes the information according to instructions in the memory module 116, and provides an output. In embodiments, the memory module 116 provides storage of data relative to the respiration signal. In embodiments, the memory module 116 stores data in a cloud-based sharing system. In embodiments, the controller 106 configured to send and receive data from the cloud-based sharing system.

In embodiments, the memory module 116 is configured as volatile and/or nonvolatile memory and, as such, may include random access memory (including SRAM, DRAM, and/or other types of RAM), flash memory, secure digital (SD) memory, registers, compact discs (CD), digital versatile discs (DVD), and/or other types of non-transitory computer-readable mediums. Depending on the particular embodiment, these non-transitory computer-readable mediums may reside within the apparatus and/or external to the apparatus. In embodiments, the memory module 116 is configured to store one or more pieces of logic, as described in more detail below. In embodiments, the controller 106 optionally includes a plurality of memory modules 116. The embodiments described herein may utilize a distributed computing arrangement to perform any portion of the logic described herein.

Embodiments of the present disclosure include logic stored on the memory module 116 that includes machine-readable instructions and/or an algorithm written in any programming language of any generation (e.g., 1GL, 2GL, 3GL, 4GL, and/or 5GL) such as, machine language that is capable of being directly executed by the processor 114, assembly language, object-oriented programming (OOP), scripting languages, microcode, etc., that may be compiled or assembled into machine readable instructions and stored on a machine readable medium. Similarly, the logic and/or algorithm may be written in a hardware description language (HDL), such as logic implemented via either a field-programmable gate array (FPGA) configuration or an application-specific integrated circuit (ASIC), and their equivalents. Accordingly, in embodiments, the logic is implemented in any conventional computer programming language, as pre-programmed hardware elements, and/or as a combination of hardware and software components. As will be described herein, logic as implemented by the controller 106 allows the system 100 to monitor and analyze a user's breathing parameters and activity. In response, the system 100 can provide a sensory output through the feedback indicator 108 relative to the respiration activity and/or respiration parameters. In some embodiments, the memory module 116 stores information associated with the user, such as previous activity data or user specific profile information, to provide a progression and/or comparison between activities or across a period of time. These and other features which may be included in the system logic will be discussed in greater detail below.

In embodiments, the controller 106 is configured to provide instructions to the user during the respiration activity, wherein the instructions correlate to a desired breathing pattern; receive the respiration signal output by the respiration sensor 104; process the respiration signal to determine a biofeedback score based on the respiration signal detected after the instructions, wherein the biofeedback score is indicative of the user's respiration signal compared to the desired breathing pattern; and provide feedback to the user through the feedback indicator 108 based on the biofeedback score.

Still referring to FIG. 1, in embodiments, the system 100 further includes a communication module 110 for communicatively coupling various modules of the system 100 with a network. The communication module 110 is communicatively coupled to the communication path 102 and can be any device capable of transmitting and/or receiving data via the network. Accordingly, in embodiments, the communication module 110 includes a communication transceiver for sending and/or receiving any wired or wireless communication. For example, the communication module 110 may include a transponder, an antenna, a modem, LAN port, Wi-Fi card, WiMax card, mobile communications hardware, near-field communication hardware, satellite communication hardware and/or any wired or wireless hardware for communicating with other networks and/or devices. In one embodiment, the communication module 110 includes hardware configured to operate in accordance with the Bluetooth wireless communication protocol. In such embodiments, the communication module 110 includes a Bluetooth send/receive module for sending and receiving Bluetooth communications to/from one or more devices (e.g., such as to the controller 106).

In some embodiments, the network includes one or more computer networks (e.g., a personal area network, a local area network, or a wide area network), cellular networks, satellite networks and/or a global positioning system and combinations thereof. Accordingly, the system 100 is communicatively coupled to the network via any suitable means, such as via wires, via a wide area network, via a local area network, via a personal area network, via a cellular network, via a satellite network, etc. Suitable local area networks include wired Ethernet and/or wireless technologies such as, for example, wireless fidelity (Wi-Fi). Suitable personal area networks include, but are not limited to, wireless technologies such as, for example, IrDA, Bluetooth, Wireless USB, Z-Wave, ZigBee, and/or other near field communication protocols. Suitable personal area networks may similarly include wired computer buses such as, for example, USB and FireWire. Suitable cellular networks include, but are not limited to, technologies such as LTE, WiMAX, UMTS, CDMA, and GSM.

In embodiments, the communication module 110 communicatively couples the system 100 and/or modules of the system 100 with an external device 118. In embodiments, the controller 106 is integrated with the external device. The external device 118 may include any device capable of allowing a user to interact with the system 100. In embodiments, the external device 118 includes mobile phones, smartphones, personal digital assistants, dedicated mobile media players, mobile personal computers, laptop computers, and/or any other mobile devices capable of being communicatively coupled with the system 100. Accordingly, in some embodiments, a user may use the system 100 as a means for communicating with others. In embodiments, the external device 118 may include communication chips, antennas, or the like to allow the telecommunications module to communicate with others via, for example, a cellular network. WiFi, or the like In embodiments, the external device 118 is any device that can provide a user interface, such as, but not limited to, a computer, a mobile device, a smart watch, and the like.

In embodiments, the external device 118 provides a display, such as a graphical user interface, and/or one or more user interface controls. The display may be, for example and without limitation, any liquid crystal display (LCD), light emitting diode (LED) display, electronic ink (e-ink) display, or the like that can display information to the user. In some embodiments, the display is configured as an interactive display that can receive user inputs (e.g., a touch screen display or the like). The user interface controls may include hardware components that receive inputs from a user and transmit signals corresponding to the inputs, such as a keyboard, a mouse, a joystick, a touch screen, a remote control, a pointing device, a video input device, an audio input device, and/or the like. In some embodiments, the system 100 is incorporated into a mobile device such that the display and the user interface controls are combined into a single device, for example a smart phone or other type mobile device. In embodiments, the feedback indicator 108 is incorporated with the external device 118. The user interface controls may allow a user to interact with the system 100, for example to initiate a respiration activity, review data, turn on and/or off a breath monitoring apparatus, or the like.

In embodiments, the external device 118 is configured to provide feedback to multiple users at the same time, such as in a group exercise or class setting. Non-limiting examples include displaying group and/or individual statistics. VR programs, orbs that are configured to light certain sections and/or colors to correspond to different users, and the like.

In embodiments, the system 100 includes one or more additional sensors 112. The additional sensor 112 can be configured to generate and output a signal based on one or more additional parameters. The additional parameters may be related to physiologic function of the user, aspects of the external environment, or aspects of the system 100 or an apparatus to which the system 100 is implemented with. Illustrative, non-limiting examples of the additional parameters include movement (e.g., acceleration, velocity, rotation), electric fields, voltage, current, magnetic field, temperature, pressure (e.g., blood pressure, barometric pressure), heart rate, heart rate variability, oxygen saturation, blood glucose, radiation, electrical conductivity, optical intensity, spatial or temporal differential (e.g., a temperature differential, a pressure differential, or a voltage differential), biological marker (e.g., a tumor marker, bacterial marker or DNA fragment), chemical composition of a substance, and a chemical reaction or a byproduct thereof. Any type of sensor configured to output a signal relative to the desired parameter may be used.

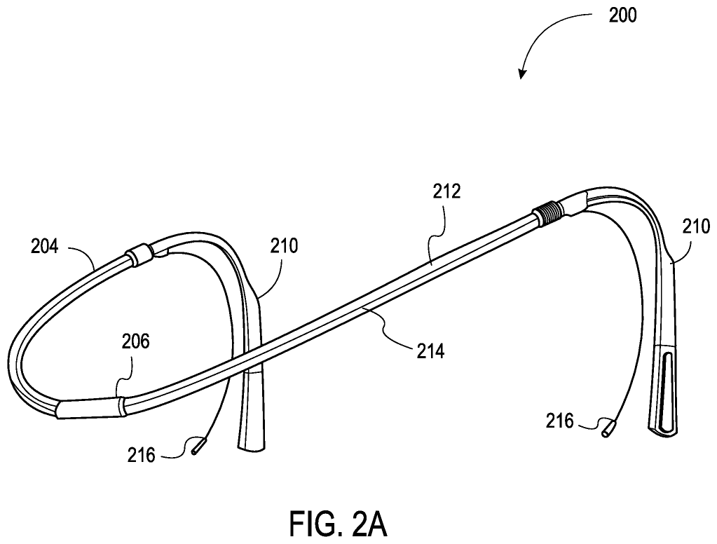

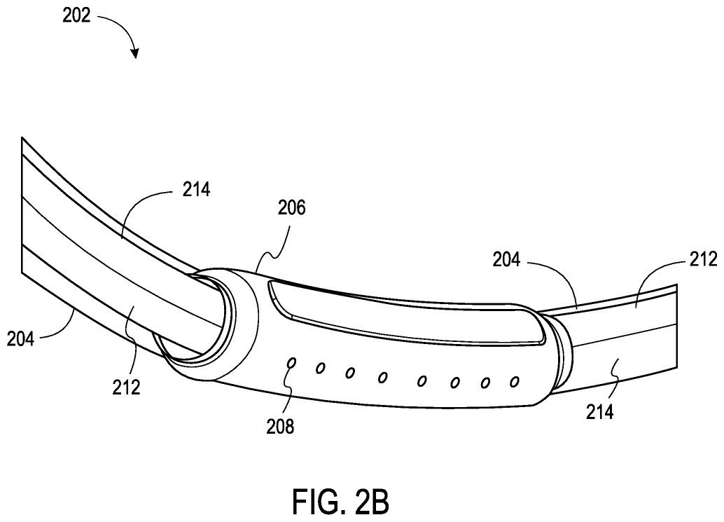

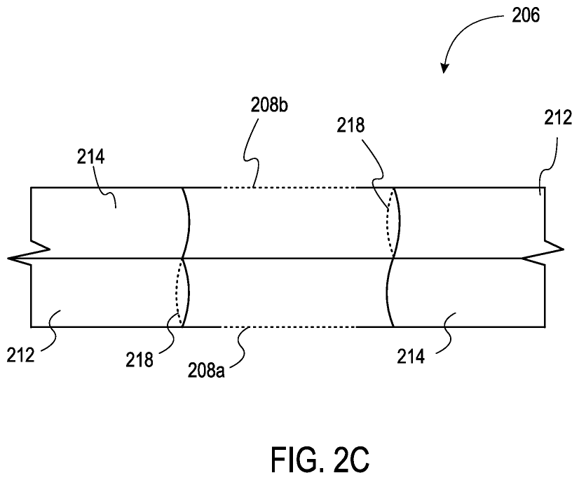

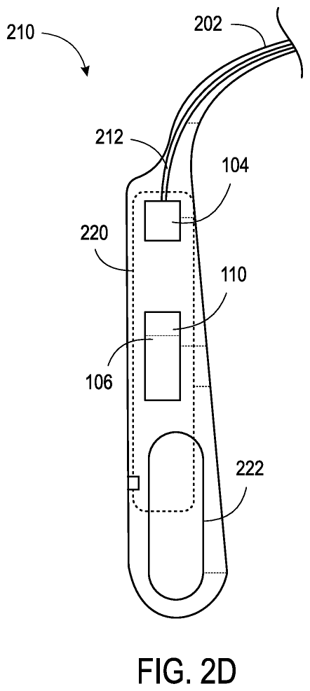

Referring now to FIG. 1 and FIGS. 2A-2D, a breath monitoring apparatus 200, incorporating system 100, is schematically depicted. In embodiments, the breath monitoring apparatus 200 is configured to deliver a respiratory sample from a user to be analyzed for evaluating respiratory parameters. In embodiments, the analysis is completed by the system 100.

In embodiments, the breath monitoring apparatus 200 includes a flexible tubing system 202, comprising one or more exterior tubes 204 and one or more cannulas 212 disposed inside the exterior tube 204. In some embodiments, the tubing system 202 does not include exterior tubing 204. The exterior tube 204 is generally a flexible, hollow tube defining a lumen. In embodiments, the exterior tube 204 is coupled to an airflow receiver 206 and/or an electronics housing 210. In embodiments, the tubing system 202 is configured to be disposable and/or replaceable, such that the tubing system can be replaced and/or cleaned if needed.

The cannula 212 may be of any appropriate construction and made from any suitable material, such as, but not limited to, polyvinyl chloride, polyurethane, polyethylene, polytetrafluoroethylene (PTFE), silicone, nylon, and the like. The cannula 212 is generally an elongate, hollow tube defining a lumen. In embodiments, the cannula is coupled to the airflow receiver 206 and/or the electronics housing 210. In embodiments, the end of the cannula 212 coupled to the electronics housing 210 is occluded. In embodiments, the end of the cannula 212 coupled to the airflow receiver defines an inlet 218 into the cannula lumen.

In embodiments, such as shown in FIG. 2B and FIG. 2C, the tubing system 202 comprises an airflow receiver 206, configured to obtain a respiratory sample from a user. In embodiments, the airflow receiver 206 is configured to be disposed between the nose and upper lip of a user. In embodiments, the airflow receiver 206 has a plurality of apertures 208 to collect a respiratory sample. In embodiments, the apertures 208 are configured to obtain a respiratory sample from a nasal passageway to evaluate a respiratory parameter associated with nasal breathing and the apertures 208b are disposed along an upper edge of the airflow receiver 206. In embodiments, the apertures 208 are configured to obtain a respiratory sample from an oral passageway to evaluate a respiratory parameter associated with oral breathing and the apertures 208a are disposed along a lower edge of the airflow receiver 206. In such embodiments, each cannula 212 is coupled to an independent respiration sensor 104 (not shown) and/or an independent electronic housing 210 to monitor and analyze both oral and nasal contributions to breathing.

In embodiments, the airflow receiver 206 is an extension of the cannula 212. In embodiments, the airflow receiver 206 comprises one or more prongs which are configured to be inserted into the nares of a user. In embodiments, the airflow receiver 206 may be configured to be inserted into the oral cavity of a user and/or placed across the oral. In embodiments, the apparatus 200 has one cannula 212 having an airflow receiver 206 which is configured to be inserted into the nares to collect nasal breathing and another cannula 212 having an airflow receiver 206 which is configured to measure breathing from the mouth.

As shown in FIG. 2B, in embodiments, the airflow receiver 206 is an elongate, hollow member, defining a lumen. In embodiments, the airflow receiver 206 is coupled to one or more exterior tubes 204. The airflow receiver 206 and exterior tube 204 are coupled using any suitable means, including, but not limited to, adhesives, friction fit, corresponding connections, and the like. In embodiments, the airflow receiver 206 and exterior tube 204 are a single, contiguous construction. In embodiments, the exterior tube 204 is a single, contiguous tube, configured to extend through the lumen of the airflow receiver 206. In embodiments, the exterior tube 204 is an individual piece independently attached to the airflow receiver 206.

In embodiments, such as shown in FIG. 2C, the airflow receiver 206 is subdivided into multiple compartments. Although FIG. 2C depicts two compartments, embodiments with other numbers of compartments are contemplated and possible. Each compartment of the airflow receiver 206 is coupled to a cannula 212. Each cannula 212 has an inlet 218 configured to transport air from the airflow receiver 206 into the cannula 212 and towards the electronic housing 210. In embodiments, the breath monitoring apparatus 200 is configured to direct airflow from the nasal orifice through a cannula 212 on one side while airflow from the oral orifice is directed through the cannula 212 on the opposite side. In embodiments, the breath monitoring apparatus 200 is configured to direct airflow from both the nasal orifice and the oral orifice through cannulas 212 on the same side.

In embodiments, the tubing system 202 is generally coupled to an electronics housing 210, as shown in FIGS. 2A and 2D, by any suitable method of connection, e.g., quick fit connectors, luer locks, friction fit, etc. In embodiments, the electronic housing 210 houses the system 100 as depicted in FIG. 1. Referring now to FIGS. 1, 2A, and 2D, in embodiments, the electronic housing 210 may include various components and/or modules of the system 100, including one or more respiration sensors 104, a controller 106, a feedback indicator 108, a communication module 110, and/or one or more additional sensors 112. In embodiments, the electronic housing 210 is configured to allow the breath monitoring apparatus 200 to be wearable as a hands-free device. For example, the breath monitoring apparatus can be wearable as a headset, a pendant configured to be worn underneath an article of clothing, attachable to an article of clothing, integrated into a hat, headband, or other apparel that allows the user to wear the device, or integrated into a face mask.

Referring still to FIGS. 2A and 2D, in embodiments, the electronic housing 210 is configured to rest behind the car of the user. In embodiments, such as shown in FIG. 2A, the breath monitoring apparatus 200 has two electronic housings 210, configured to rest behind each car of the user. In such embodiments, each electronic housing 210 includes modules and/or components of the system 100, shown in FIG. 1, including the respiration sensor 104. In such embodiments, the respiration sensor 104 disposed in the first electronic housing 210 is configured to output a respiration signal indicative of a respiration parameter related to oral breathing. Similarly, the respiration sensor 104 disposed in the second electronic housing 210 is configured to output a respiration signal indicative of a respiration parameter related to nasal breathing. Such embodiments may include one or more controllers 106. In embodiments having a plurality of controllers 106, one of the controllers 106 is configured to communicate with the second controller 106 using circuit to circuit communication, while the second controller 106 is configured to process, analyzed and communicate the data.

In embodiments, the electronic housing 210 is configured to be worn as an car clip. In embodiments, different modules and/or components of the system 100 are disposed in various positions within the breath monitoring apparatus 200. For example, and not as a limitation, the one or more respiration sensors 104 may be positioned proximate to the nose and/or mouth, such as in the airflow receiver 206, or as an adhesive patch (not shown).

In embodiments, such as shown in FIG. 2D the tubing system 202 is coupled to the electronic housing 210. In embodiments, an occluded end of cannula 212 is coupled to the electronic housing 210. In embodiments, the cannula 212 is occluded by a respiration sensor 104, such as a pressure transducer. The respiration sensor 104 is coupled to the cannula 212 using any suitable means, including barbs, nipples, and the like. In embodiments, the cannula 212 is disposed such that it is near or touching the respiration sensor 104. In embodiments, the respiration sensor is 104 is a pressure transducer, configured to measure a pressure change in the cannula 212 as a result of a respiration activity. In embodiments, the controller 106 and/or communications module 110 are disposed within the electronic housing 210. In embodiments, the electronic housing 210 has a power source 222. In embodiments, the power source may be any suitable power source, including but not limited to, a rechargeable battery, a disposable battery, an external power connection and the like.

Referring now to FIGS. 1 and 2A-2D, in embodiments, the apparatus 200 further includes one or more feedback indicators 108. In embodiments, the tubing system 202 may include visual feedback for the user, e.g. a light 214 embedded in the lumen of the exterior tube 204 or placed on the outside of the exterior tube 204. In embodiments, the light is a light tube and/or one or more light emitting diodes (LEDs). In embodiments, the light 214 is communicatively coupled to the controller 106. In embodiments, the color of the light 214 indicates adherence to a specific breathing pattern. For example, the light 214 may flash green when a user's breathing matches a targeted breathing pattern or the light 214 may flash red when a user's breathing is outside the target breathing pattern.

In embodiments, such as shown in FIG. 2A, the breath monitoring apparatus 200 further includes support structures 216, configured to keep the breath monitoring apparatus 200 in a desired position. As used herein, the term “support structure” may refer to any device or structure configured to aid in making the breath monitoring apparatus 200 wearable by a user, for example, on a user's head, face, neck, cars, nose or the like, or any combination thereof. In embodiments, the support structure 216 is a shape memory material, for example, metals, polymers, plastics, combinations thereof and the like that retain the shape. In such embodiments, the support structures 216 are coupled to the tubing system 202, such that the support structures 216 pull the tubing system 202 to allow a user to keep the breath monitoring apparatus 200 in a correct location, for example between the nose and mouth of the user. In embodiments, the support structure 216 is configured to be customizable for an individual user, such as by constructing the support structure out of a malleable wire, which is capable of being conformed to a user's car shape.

In embodiments, the breath monitoring apparatus 200 includes headphones or any suitable apparatus configured to provide auditory feedback to the user, such as, for example a speaker disposed within the electronics housing 210 or coupled to the support structure 216.

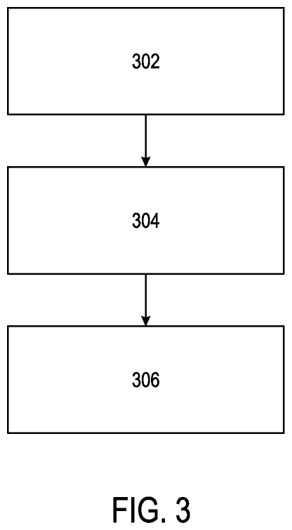

Referring now to FIG. 3, a flow chart depicting a method 300 is schematically depicted. It is noted that though a number of steps are generally depicted, a fewer or greater number of steps, taken in any order, are contemplated without departing from the scope of the present disclosure. In an illustrative, non-limiting example, the method is performed by the system 100 depicted in FIG. 1 and/or the breath monitoring apparatus 200, depicted in FIGS. 2A-2D. It is noted that various portions of the method 300 disclosed herein may be performed by a processor of an external device 118 (depicted in FIG. 1) such as through an application associated with the breath monitoring apparatus 200 (depicted in FIGS. 2A-2D) on the external device 118. In some embodiments, various portions of the method 300 are performed by different computing devices, such as in a distributed computing environment.

At block 302, the process begins by sensing and recording data (e.g., pressure change corresponding to breathing) relating to a respiration parameter of the user. In some embodiments, the data may be filtered, analyzed, or otherwise processed to infer one or more respiratory parameters or activities corresponding to the user. This data from the one or more respiration sensors 104 may be recorded by the processor 114 and stored in the memory module 116.

Referring now to FIGS. 1, 2A-2D and 3, in embodiments, the respiration sensor 104 does not sense a signal until a respiration activity is initiated by the user, such as selecting a specific program or activity within the application associated with the breath monitoring apparatus 200. In such embodiments, the user may communicate remotely with the system 100 using the external device 118 to activate a targeted breathing pattern and/or respiration rate. In other embodiments, the respiration activity will begin at a scheduled time, such as a planned group breathing activity. In such embodiments, the respiration parameter is continuously detected and the respiration signal output for the duration of the respiration activity.

A target breathing pattern is customizable and can be designed to accomplish a variety of health or wellness goals, including fitness, relaxation, physical therapy, and the like. Target breathing patterns may include guidance for duration of inhalation, inspiratory hold, exhalation, and/or expiratory hold. For example, in one embodiment, the target breathing pattern may comprise exhaling twice as long as inhaling. In another example, a target breathing pattern may comprise inhaling for a specified number of seconds, and then exhaling for a specified number of seconds. For example, the target breathing pattern may comprising inhaling for 5 seconds and exhaling for 5 seconds, or inhaling for 5 seconds and exhaling for 10 seconds, etc. The target breathing pattern could be box breathing (e.g., inhale for four seconds, hold for four seconds, exhale for four seconds, hold for four seconds). The skilled artisan will appreciate that these embodiments are exemplary in nature and that any number of suitable target breathing patterns may be designated.

In embodiments, the target breathing pattern may include a target inhalation time, target hold time, and/or target exhalation time. In embodiments, the target breathing pattern may include a minimum expiratory energy and/or minimum inspiratory energy. In other embodiments, the target breathing pattern may include the user reaching peak expiratory or inspiratory flow within a certain time. Targeted breathing patterns may also include preset programs that correspond with breathing pattern targets for specific activities (e.g., Yogi 2:1 breathing). In another embodiment, the system 100 can include preset targets and the ability to program custom breathing pattern targets. In another embodiment, the breathing pattern target may change temporally. e.g., breathing at a rate of 10 bpm for 2 minutes, then 7.5 bpm for 2 minutes, etc. In another embodiment, the target may be the movement of different breathing muscles at different times. For example, a user may wish to learn lateral breathing for Pilates, in which the abdomen is kept flat while the ribs expand laterally. In this case, the target would be minimal abdominal movement during breathing (which could be measured using a plethysmograph). In other embodiments, the user may wish to reduce the use of accessory muscles during breathing. In this case, the target would be to not activate those muscles (which could be measured using EMG). In embodiments, the target breathing pattern may include inhaling with the nose and exhaling with the mouth. In other embodiments, the targeted breathing pattern may involve inhaling and exhaling through the nose. In other embodiments, the targeted breathing pattern can be programmed to mimic another user's breathing pattern (i.e., an instructor). In such an embodiment, the instructor's breathing could be measured live, or it could be pre-recorded and imported into the preset programs.

In embodiments, the target breathing pattern is communicated to the user via audio cues, tactile cues, visual cues, and/or a combination thereof. Once the target breathing pattern is defined, the system 100 provides instructions or cues to the so that the user can attempt to follow the target breathing pattern. In embodiments, the instructions correlate with a specific respiration activity (e.g., specific steps, such as inhalation, exhalation, holds, natural breathing, short hold, long hold, etc.). For example, one sound could be played during the desired inhalation and a different sound could be played during the desired exhalation time. In another embodiment, verbal instructions, such as “breathe in” are provided to the user at the appropriate time. Other audio instructions are possible. In embodiments, the instructions or cues could also be delivered visually as described above. In embodiments, each program comprises multiple sets of instructions and/or respiration activities within a single program.

Referring jointly to FIGS. 1 and 3, at block 304, the user's data relative to the respiration signal indicative of the respiration parameter is analyzed to determine and/or infer compliance with the program. In embodiments, analysis is also conducted within a program to determine the compliance of the user to the previous instruction and/or respiration activity. In embodiments, this analysis allows for real-time adaptation of the program and/or respiration activity. In embodiments, the controller 106 is configured to adapt the program based on the analyzed parameters. For example, the controller 106 may be configured to adapt the target time for a respiration activity correlated with a change in breathing phase (e.g., inhalation to exhalation) such that the target time of the next phase does not start until the user's breathing pattern actually enters that phase and the previous phase ends. For example, if the target inhalation time within a given program is 4 seconds, followed by a 4 second hold, but the user inhales for 4.5 seconds, the controller 106 will adjust the program such that the timer for the hold starts at 4.5 second into the session (end of actual inhalation) rather than at the end of target inhalation (4 seconds).

In some embodiments, the controller 106 receives instructions from the communication module 110 by the user from one or more external devices 118. In embodiments, the controller 106 uses the received data to adjust the program or patterns. For example, if the external device 118 and/or an additional sensor 112 collect a heart rate above a certain level, the controller 106 could adjust the targeted breathing program based on the heart rate data. In another embodiment, if the collected data indicates an improving fitness profile, the controller 106 can adjust the targeted breathing pattern appropriately. For example, the system 100 could be configured to allow one respiratory rate for slow walking pace and a higher respiratory rate for a faster walking pace. In another embodiment, the breathing target could be determined using the user's current respiration rate. In such embodiments, the respiration sensor 104 measures the user's control parameter (e.g., inhalation time) and, in real-time, updates the target parameter (e.g., exhalation time). For example, if the target were to have an exhalation time twice as long as inhalation time, during one breath the user's inhale time might be 2 seconds, in which case the target for the next exhalation would be 4 seconds. The next inhalation might then be 2.5 seconds in which case the target for the subsequent exhalation would be 5 seconds.

Still referring jointly to FIGS. 1 and 3, at block 306, feedback is provided to the user through the feedback indicator 108. In embodiments, the feedback is transmitted from the controller 106. In embodiments, the feedback is transmitted from a processor in an external device 118. In embodiments, feedback is transmitted from both the controller 106 and an external device 118.

Real-time feedback can be given as guidance to help a user achieve a desired breathing pattern, or as encouragement while the program is in progress. In embodiments, real-time feedback is generated based on the calculation of a breathing score of the user. Feedback given after the completion of a program could be given as a score, as described below. Feedback can be auditory, visual, and/or tactile. In embodiments, feedback can be given as cues or guidance to achieve a target breathing pattern. Other embodiments include feedback regarding individual breathing patterns and/or breathing rate changes associated with other data, including, but not limited to: heart rate variability, physical activity minutes, step count, heart rate, weight, blood pressure, fatigue, mood, etc.

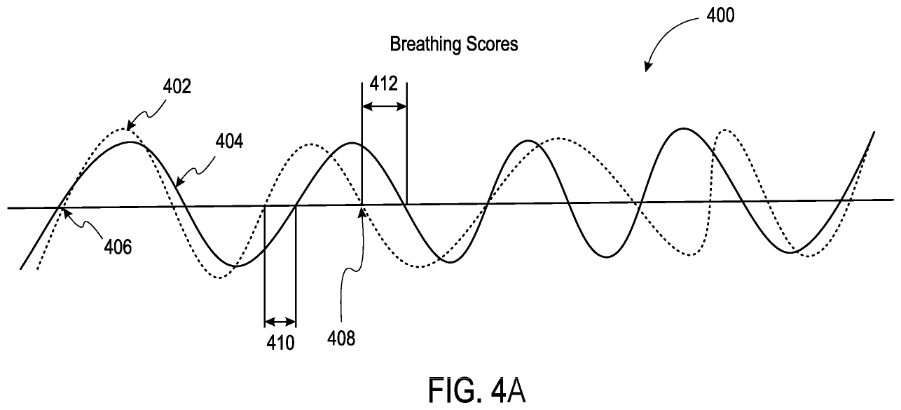

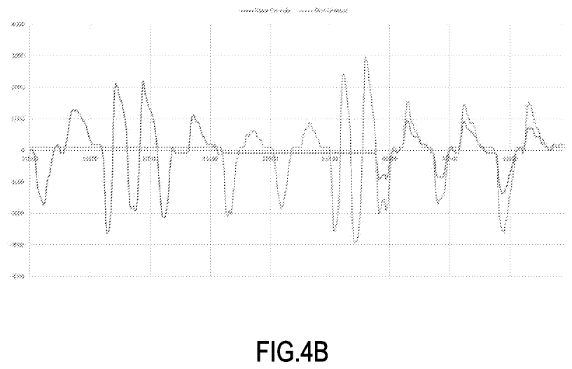

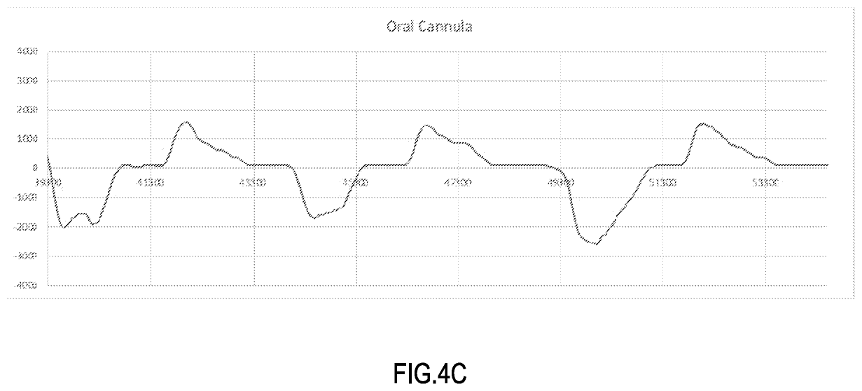

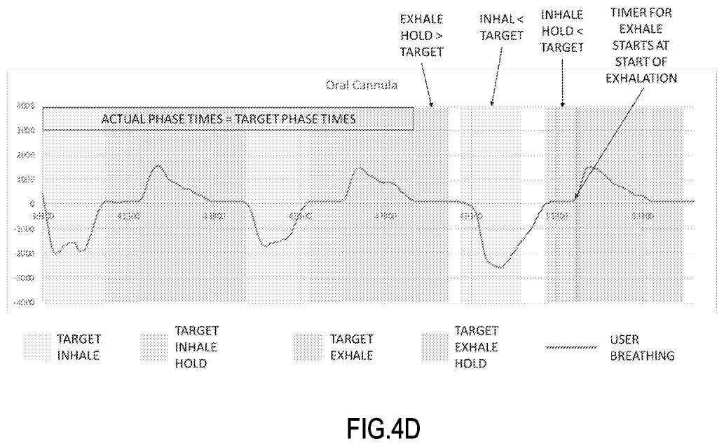

In some embodiments, feedback is given to the user a biofeedback score. In embodiments, the feedback can also include scoring or reviews of how well as user is meeting a target breathing pattern, such as represented in FIGS. 4C and 4D. A biofeedback score can be calculated by the system in any number of ways. In embodiments, the biofeedback score is calculated as the percentage. For example, the biofeedback score could be calculated by dividing the number of successful transitions a user has between breathing phases in a breathing program by the total number of breath transitions within the program. To prevent false transitions between breathing phases, the breath monitoring apparatus is calibrated to store the output value of the breathing sensor during periods of no breathing. In embodiments the calibration value is manually set. In other embodiments, the system is configured to run an automatic calibration routine that adjusts the calibration values. Threshold values for breathing phases will be empirically set at a value to avoid false triggers, dependent on the design of the device.

In some embodiments, the biofeedback score is calculated as the percent contribution of nasal and/or oral breathing, such as shown in FIG. 4B. In embodiments, the area under the curve (AUC) is calculated for both a nasal and oral sensor. The percent nasal breathing contribution is calculated by dividing the nasal AUC by the total AUC. Similarly, the percent oral breathing contribution is calculated by dividing the oral AUC by the total AUC. In some embodiments, a scaling factor may need to be applied so that the total AUC is proportional to the total breath volume. In some embodiments, the biofeedback score is evaluated as a binary variable. For example, if oral breathing is above a threshold value, the variable is considered on. If oral breathing is below the threshold value, the variable is considered off. The same system is also used to evaluate nasal breathing.

In some embodiments where a user is using an external device 118 to perform portions of a method, a user interface of the external device displays the biofeedback score. In embodiments, the biofeedback score is normalized to 100 and provides a range of categories based on percent adherence to the programs (e.g., 0-20, 21-40, 41-60, 61-80, and 81-100). In embodiments, each category is given a descriptive label relating to the user's success. The score represents how well the user matched the desired breathing pattern. This score is communicated to the user via feedback on the user interface.

In embodiments, the biofeedback score is calculated as the mean of the absolute differences or the root mean square (RMS) between the desired and actual start of inhalation and exhalation. In other embodiments, the biofeedback breathing score compares the percent through each breathing phase in the desired breathing pattern to the percent through each breathing phase in actual breathing pattern. This score is also calculated as RMS. In other embodiments, the score is presented as a respiratory rate variability, which is calculated as the RMS of the time from the inhalation of one breath to the time of inhalation to the next breath. Alternatively, this could be calculated as the standard deviation of each breath period.

Referring to FIGS. 1, 2A-2D and 3, in embodiments, the biofeedback score may be indicated by changing the color of the light 214. In addition to the score, the system 100 also provides information about the user's performance, e.g., length of the session, etc., and combines this information with other collected and stored data to track changes and improvements over time. For example, a user wanting to increase heart rate variability over time would be able to monitor their heart rate and track this information relative to their stored respiratory activity data.

FIG. 4A shows a graph 400 of desired breathing patterns vs. actual breathing patterns that can be used to calculate breathing scores. The graphed line 402 is representative of the desired breathing signal. The graphed line 404 is the actual breathing signal. The beginning of a user's inhalation is indicated at 406, while the start of exhalation is indicated at 408. The measured distance 410 is the difference between the desired and actual start of inhalation. The measured distance 412 is the difference between the desired and actual start of exhalation. The start of inhalation occurs when the breathing signal shifts from negative to positive. The start of exhalation occurs when signal shifts from positive to negative.

Embodiments of the present disclosure can be described with reference to the following aspects.

In a first aspect of the present disclosure, alone or in combination with any other aspect disclosed herein, a breath monitoring apparatus is provided, having one or more respiration sensors configured to output a respiration signal indicative of a respiration parameter of a user during a respiration activity; a feedback indicator configured to provide a sensory output to a user relating to the respiration signal; and a controller communicatively coupled to the respiration sensor and the feedback indicator, the controller configured to: provide instructions to the user during the respiration activity, wherein the instructions correlate to a desired breathing pattern; receive the respiration signal output by the respiration sensor; analyze the respiration signal to determine a biofeedback score based on the respiration signal detected after the instructions, wherein the biofeedback score is indicative of the user's respiration signal compared to the desired breathing pattern; and provide feedback to the user through the feedback indicator based on the biofeedback score, wherein the breath monitoring apparatus is configured to be wearable by the user.

In a second aspect of the present disclosure, alone or in combination with any other aspect disclosed herein, a breath monitoring apparatus is provided, having a tubing system, said tubing system comprising: an airflow receiver, configured to obtain a respiratory sample from the user; and one or more cannulas configured to transport the respiratory sample to the respiration sensor.

In a third aspect of the present disclosure, alone or in combination with any other aspect disclosed herein, a breath monitoring apparatus is provided, having an airflow receiver configured to obtain the respiratory sample from both oral breathing and nasal breathing of the user.

In a fourth aspect of the present disclosure, alone or in combination with any other aspect disclosed herein, a breath monitoring apparatus is provided, having a first respiration sensor configured to output a respiration signal indicative of oral breathing; and a second respiration sensor configured to output a respiration signal indicative of nasal breathing.

In a fifth aspect of the present disclosure, alone or in combination with any other aspect disclosed herein, a breath monitoring apparatus is provided, wherein the one or more cannulas are configured to build pressure in response to the respiratory sample.

In a sixth aspect of the present disclosure, alone or in combination with any other aspect disclosed herein, a breath monitoring apparatus is provided, wherein the tubing system is configured to be positioned between the nose and mouth of the user.

In a seventh aspect of the present disclosure, alone or in combination with any other aspect disclosed herein, a breath monitoring apparatus is provided, wherein the one or more respiration sensors are pressure transducers.

In an eighth aspect of the present disclosure, alone or in combination with any other aspect disclosed herein, a breath monitoring apparatus is provided, wherein the one or more cannulas are occluded by the respiration sensor.

In an ninth aspect of the present disclosure, alone or in combination with any other aspect disclosed herein, a breath monitoring apparatus is provided, wherein the feedback indicator is disposed in the tubing system.

In a tenth aspect of the present disclosure, alone or in combination with any other aspect disclosed herein, a breath monitoring apparatus is provided, wherein the feedback indicator is a light tube or LED.

In an eleventh aspect of the present disclosure, alone or in combination with any other aspect disclosed herein, a breath monitoring apparatus is provided, wherein the light tube or LED changes color based on the biofeedback score.

In a twelfth aspect of the present disclosure, alone or in combination with any other aspect disclosed herein, a breath monitoring apparatus is provided, wherein the apparatus is wearable as a headset.

In a thirteenth aspect of the present disclosure, alone or in combination with any other aspect disclosed herein, a breath monitoring apparatus is provided, wherein the controller and respiration sensor are disposed within an electronic housing.

In a fourteenth aspect of the present disclosure, alone or in combination with any other aspect disclosed herein, a system for monitoring respiration is provided, having a respiration sensor configured to output a respiration signal indicative of a respiration parameter of a user during a respiration activity; a feedback indicator configured to provide a sensory output to a user relating to the respiration signal; and a controller coupled to the respiration sensor and the feedback indicator, the controller configured to: provide instructions to the user during the respiration activity, wherein the instructions correlate to a desired breathing pattern; receive the respiration signal detected by the respiration sensor; analyze the respiration signal to determine a biofeedback score based on the respiration signal detected after the instructions, wherein the biofeedback score is indicative of the user's respiration signal compared to the desired breathing pattern; and provide feedback to the user through the feedback indicator based on the biofeedback score.

In a fifteenth aspect of the present disclosure, alone or in combination with any other aspect disclosed herein, a system for monitoring respiration is provided, wherein the feedback indicator is a light tube or LED.

In a sixteenth aspect of the present disclosure, alone or in combination with any other aspect disclosed herein, a system for monitoring respiration is provided, wherein the light tube or LED changes color based on the biofeedback score.

In a seventeenth aspect of the present disclosure, alone or in combination with any other aspect disclosed herein, a system for monitoring respiration is provided, wherein the instructions are provided to the user on an external device.

In an eighteenth aspect of the present disclosure, alone or in combination with any other aspect disclosed herein, a system for monitoring respiration is provided, wherein the external device is a mobile device.

In a nineteenth aspect of the present disclosure, alone or in combination with any other aspect disclosed herein, a system for monitoring respiration is provided, wherein the respiration sensor is a pressure transducer.

In a twentieth aspect of the present disclosure, alone or in combination with any other aspect disclosed herein, a method for monitoring respiration using the apparatus and/or system of any of the aspects disclosed herein is provided, the method comprising providing instructions to a user during a respiration activity, wherein the instructions correlate to a desired breathing pattern; sensing a respiration parameter using a respiration sensor configured to output a respiration signal indicative of the respiration parameter of the user during the respiration activity; analyzing the respiration signal to determine a biofeedback score based on the respiration signal detected after the instructions, wherein the biofeedback score is indicative of the user's respiration signal compared to the desired breathing pattern; and providing feedback to the user through a feedback indicator configured to provide a sensory output to the user based on the biofeedback score.

In a twenty-first aspect of the present disclosure, alone or in combination with any other aspect disclosed herein, a method for providing feedback using the apparatus and/or system of any of the aspects disclosed herein is provided, the method comprising providing instructions to a user during a respiration activity, wherein the instructions correlate to a desired breathing pattern; sensing a respiration parameter using a respiration sensor configured to output a respiration signal indicative of the respiration parameter of the user during the respiration activity; analyzing the respiration signal to determine a biofeedback score based on the respiration signal detected after the instructions, wherein the biofeedback score is indicative of the user's respiration signal compared to the desired breathing pattern; and providing feedback to the user through a feedback indicator configured to provide a sensory output to the user based on the biofeedback score.

In a twenty-second aspect of the present disclosure, alone or in combination with any other aspect disclosed herein, a method for operating the apparatus and/or system of any of the aspects disclosed herein is provided, the method comprising initiating a respiration activity, providing instructions during the respiration activity, wherein the instructions correlate to a desired breathing pattern; sensing a respiration parameter using a respiration sensor configured to output a respiration signal indicative of the respiration parameter of the user during the respiration activity; analyzing the respiration signal to determine a biofeedback score based on the respiration signal detected after the instructions, wherein the biofeedback score is indicative of the user's respiration signal compared to the desired breathing pattern; and providing feedback to the user through a feedback indicator configured to provide a sensory output to the user based on the biofeedback score.

All documents cited are incorporated herein by reference in their entirety; the citation of any document is not to be construed as an admission that it is prior art with respect to the present invention.

It should now be understood that the systems and methods described herein allow for monitoring of breathing in real time. Furthermore, the systems and methods described herein allow for the monitoring of the breathing patterns across time. By allowing for real-time data collection, users can become more in tune with health and improve fitness levels.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A breath monitoring apparatus comprising:
one or more respiration sensors configured to output a respiration signal indicative of a respiration parameter of a user during a respiration activity;
a feedback indicator configured to provide a sensory output to a user relating to the respiration signal; and
a controller communicatively coupled to the respiration sensor and the feedback indicator, the controller configured to:
provide instructions to the user during the respiration activity, wherein the instructions correlate to a desired breathing pattern;
receive the respiration signal output by the respiration sensor;
analyze the respiration signal to determine a biofeedback score based on the respiration signal detected after the instructions, wherein the biofeedback score is indicative of the user's respiration signal compared to the desired breathing pattern; and
provide feedback to the user through the feedback indicator based on the biofeedback score,
wherein the breath monitoring apparatus is configured to be wearable by the user.

2. The breath monitoring apparatus of claim 1, further comprising a tubing system, said tubing system comprising:
an airflow receiver, configured to obtain a respiratory sample from the user; and
one or more cannulas configured to transport the respiratory sample to the respiration sensor.

3. The breath monitoring apparatus of claim 2, wherein the airflow receiver is configured to obtain the respiratory sample from both oral breathing and nasal breathing of the user.

4. The breath monitoring apparatus of claim 3, wherein the one or more respiration sensors comprise:
a first respiration sensor configured to output a respiration signal indicative of oral breathing; and
a second respiration sensor configured to output a respiration signal indicative of nasal breathing.

5. The breath monitoring apparatus of claim 2, wherein the one or more cannulas are configured to build pressure in response to the respiratory sample.

6. The breath monitoring apparatus of claim 2, wherein the tubing system is configured to be positioned between the nose and mouth of the user.

7. The breath monitoring apparatus of claim 1, wherein the one or more respiration sensors are pressure transducers.

8. The breath monitoring apparatus of claim 2, wherein the one or more cannulas are occluded by the respiration sensor.

9. The breath monitoring apparatus of claim 2, wherein the feedback indicator is disposed in the tubing system.

10. The breath monitoring apparatus of claim 9, wherein the feedback indicator is a light tube or LED.

11. The breath monitoring apparatus of claim 10, wherein the light tube or LED changes color based on the biofeedback score.

12. The breath monitoring apparatus of claim 1, wherein the apparatus is wearable as a headset.

13. The breath monitoring apparatus of claim 1, wherein the controller and respiration sensor are disposed within an electronics housing.

14. A system for monitoring respiration comprising:
a respiration sensor configured to output a respiration signal indicative of a respiration parameter of a user during a respiration activity;
a feedback indicator configured to provide a sensory output to a user relating to the respiration signal; and
a controller coupled to the respiration sensor and the feedback indicator, the controller configured to:
provide instructions to the user during the respiration activity, wherein the instructions correlate to a desired breathing pattern;
receive the respiration signal detected by the respiration sensor;
analyze the respiration signal to determine a biofeedback score based on the respiration signal detected after the instructions, wherein the biofeedback score is indicative of the user's respiration signal compared to the desired breathing pattern; and
provide feedback to the user through the feedback indicator based on the biofeedback score.

15. The system of claim 14, wherein the feedback indicator is a light tube or LED.

16. The system of claim 15, wherein the light tube or LED changes color based on the biofeedback score.

17. The system of claim 14, wherein the instructions are provided to the user on an external device.

18. The system of claim 17, wherein the external device is a mobile device.

19. The system of claim 14, wherein the respiration sensor is a pressure transducer.

20. A method for monitoring respiration using the apparatus of claim 1, the method comprising:
providing instructions to a user during a respiration activity, wherein the instructions correlate to a desired breathing pattern;
sensing a respiration parameter using a respiration sensor configured to output a respiration signal indicative of the respiration parameter of the user during the respiration activity;
analyzing the respiration signal to determine a biofeedback score based on the respiration signal detected after the instructions, wherein the biofeedback score is indicative of the user's respiration signal compared to the desired breathing pattern; and
providing feedback to the user through a feedback indicator configured to provide a sensory output to the user based on the biofeedback score.
